(19) United States
(12) Patent Application Publication
(10) Pub. No.: US 2024/0219708 A1
(43) Pub. Date: Jul. 4, 2024

(54) METHOD FOR MANUFACTURING MULTIPLE FILTERS WITH FABRY-PEROT CAVITY STRUCTURE, MULTISPECTRAL TRANSMISSION FILTER ARRAY AND MULTISPECTRAL TRANSMISSION FILTER STRUCTURE

(71) Applicants: LITE-ON SINGAPORE PTE. LTD., Singapore (SG); Singapore University of Technology and Design, Singapore (SG)

(72) Inventors: RUI-TAO ZHENG, Singapore (SG); SIN-HENG LIM, SINGAPORE (SG); WUI-PIN LEE, Singapore (SG); SRINIVASAN LAKSHMANAN CHETTIAR, SINGAPORE (SG); Md Abdur Rahman, Singapore (SG); Joel Kwang Wei Yang, Singapore (SG)

(21) Appl. No.: 18/391,558

(22) Filed: Dec. 20, 2023

(30) Foreign Application Priority Data
Dec. 30, 2022 (SG) 10202260647U

(51) Int. Cl. G02B 26/00; G02B 1/00; G02B 5/20; G02B 5/28

(52) U.S. Cl. CPC G02B 26/001; G02B 1/002; G02B 5/201; G02B 5/286; G02B 2207/101

(57) ABSTRACT

A method of manufacturing a plurality of optical filters with Fabry-Pérot (F-P) cavity structures, comprising: providing a carrier; disposing a first reflection layer on the carrier; disposing a first pattern optical layer and a second pattern optical layer on the surface of the first reflection layer; the first pattern optical layer includes a plurality of first nanostructures and a second pattern optical layer includes a plurality of second nanostructures; reflowing the first optical film layer and the second optical film layer to form a first film layer and a second film layer; and coating a second reflection layer on the first film layer and the second film layer.

### CROSS-REFERENCE TO RELATED PATENT APPLICATION

This application claims the benefit of priority to Singapore Patent Application No. 10202260647U, filed on Dec. 30, 2022. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an optical filter, and more particularly to an optical filter with Fabry-Pérot (F-P) cavity.

### BACKGROUND OF THE DISCLOSURE

In the related art, image sensors are an integral part of today's electronics, especially in digital cameras and mobile phones. Conventionally, both charged coupled devices (CCDs) and complementary metal oxide semiconductor (CMOS) image sensors are used as Si-based image sensors, but the CMOS image sensors are generally more advantageous than the CCD image sensors due their compactness, cost-efficiency, and low power consumption.

In order to exhibit color sensitivity, color filters of the image sensors are generally equipped with photodiodes. These color filters are usually composed of organic dye filters. However, due to lack of durability in high temperature, high-cost fabrication processes are highly impractical for multispectral imaging where more than three primary colors are required.

Therefore, F-P cavity structures, which include an intermediate dielectric layer sandwiched by two metallic reflectors, became a promising candidate for multispectral transmission filters due to their superior narrow band full width at half maximum (FWHM). In F-P cavity structures, colors can be tuned by controlling the thickness of the intermediate dielectric layer. Therefore, methods capable of exerting control over the thickness of the intermediate dielectric layers and fabrication of multispectral filters in a single chip have become very important in the relevant industry.

### SUMMARY OF THE DISCLOSURE

In response to the above-referenced technical inadequacies, the present disclosure provides a method of manufacturing a plurality of optical filters with Fabry-Pérot (F-P) cavity structures and a multispectral transmission filter array.

In order to solve the above-mentioned problems, one of the technical aspects adopted by the present disclosure is to provide a method of manufacturing a plurality of optical filters with Fabry-Pérot (F-P) cavity structures, comprising: providing a carrier; disposing a first reflection layer on the carrier; disposing a first pattern optical layer and a second pattern optical layer on the surface of the first reflection layer; wherein the first pattern optical layer includes a plurality of first nanostructures and a second pattern optical layer includes a plurality of second nanostructures, a first pitch being formed between two adjacent first nanostructures, a second pitch being formed between two adjacent second nanostructures; reflowing the first optical film layer and the second optical film layer to form a first film layer and a second film layer; and coating a second reflection layer on the first film layer and the second film layer.

In order to solve the above-mentioned problems, another one of the technical aspects adopted by the present disclosure is to provide multispectral transmission filter array, comprising: a carrier, a first reflection layer, an optical layer and a second reflection layer. The first reflection layer disposed on the carrier. The optical layer disposed on the carrier, wherein a first color pixels, a second color pixels, formed by the optical layer with difference thickness, and interpixel boundary formed between thereof. The second reflection layer disposed on the profile of the optical layer.

In order to solve the above technical problems, another one of the technical aspects adopted by the present disclosure is to provide a multispectral transmission filter structure, comprising: a carrier, a first transmission filter and a second transmission filter. The carrier has a first sensing region and a second sensing region. The first transmission filter includes a first bottom reflective layer, a first optical structure and a first top reflective layer. The first bottom reflective layer is disposed in the first sensing region. The first optical structure includes a first recess having a first depth. The first top reflection layer is disposed on the first optical structure. The second transmission filter is adjacent to the first transmission filter and includes a second bottom reflection layer, a second optical structure and a second top reflection layer. The second bottom reflection layer is disposed in the second sensing region. The second optical structure includes a second recess having a second depth. The second top reflection layer is disposed on the second optical structure. The first depth is different from the second depth.

In addition, adjusting the thickness of the optical layer is equivalent to adjusting thickness of Fabry-Pérot (F-P) cavity structures which will modulate peak wavelength of optical filter. For example, the optical filter can span both visible to near infra-red.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 to FIG. 5 are schematic diagrams corresponding to a embodiment of a manufacturing method of the present disclosure;

FIG. 6 to FIG. 8, are schematic diagrams corresponding to patterning step of a manufacturing method of the present disclosure;

FIG. 9A to FIG. 9C are schematic diagrams of patterns according to an embodiment of the present disclosure;

FIG. 10 is a schematic diagram showing the relationship between the thickness of the reflection layer and the number of nanostructures of the present disclosure;

FIG. 11 is a schematic diagram showing the relationship between the thickness, transmittance and wavelength of the reflection layers of an embodiment of the present disclosure; and

FIG. 12 is a schematic diagram according to an embodiment of a multispectral transmission filter structure of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

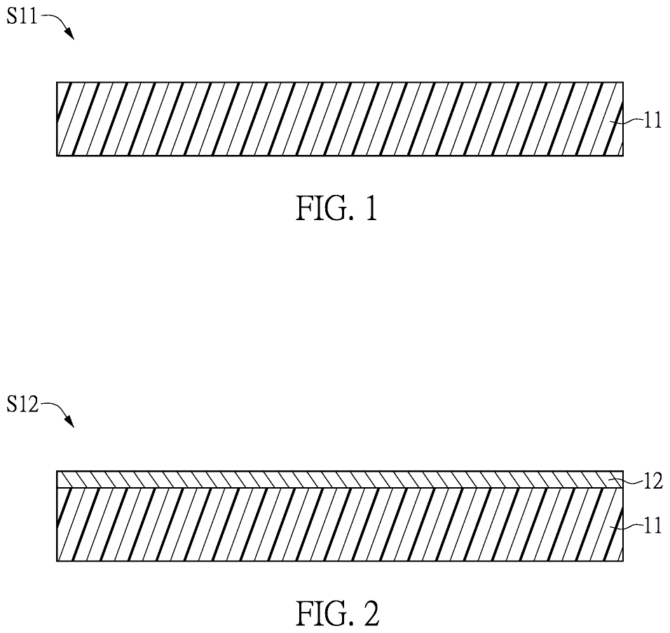

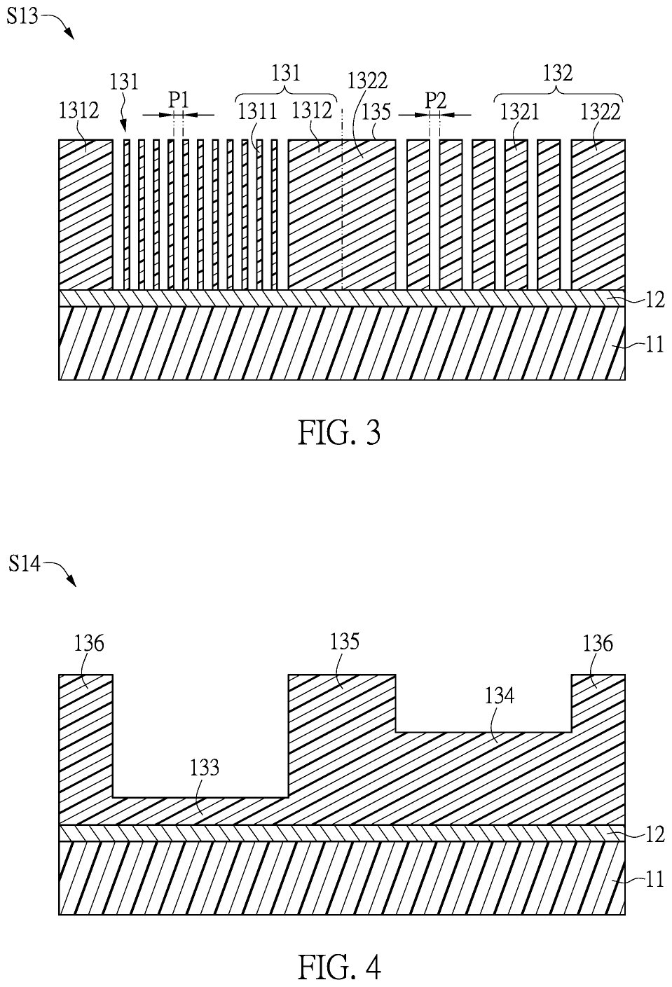

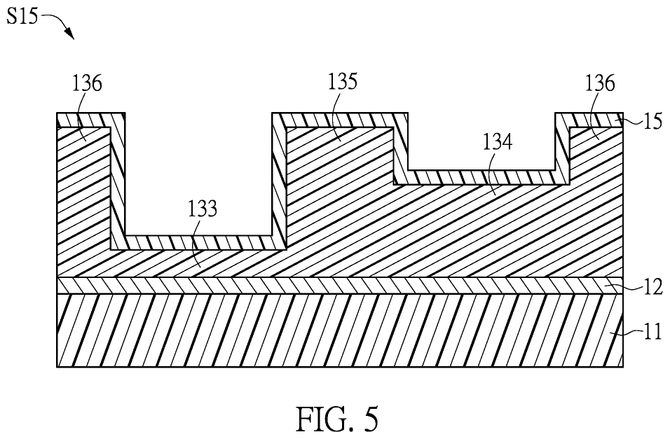

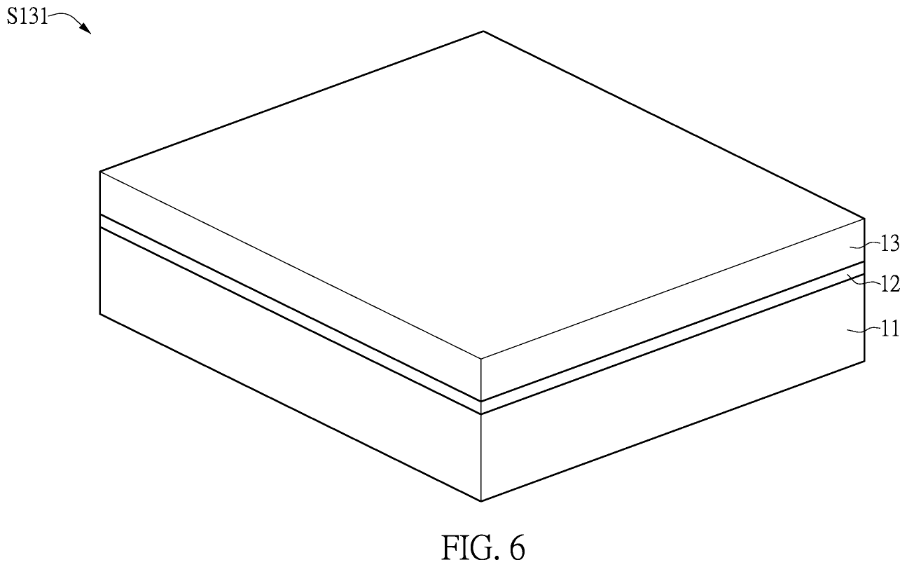

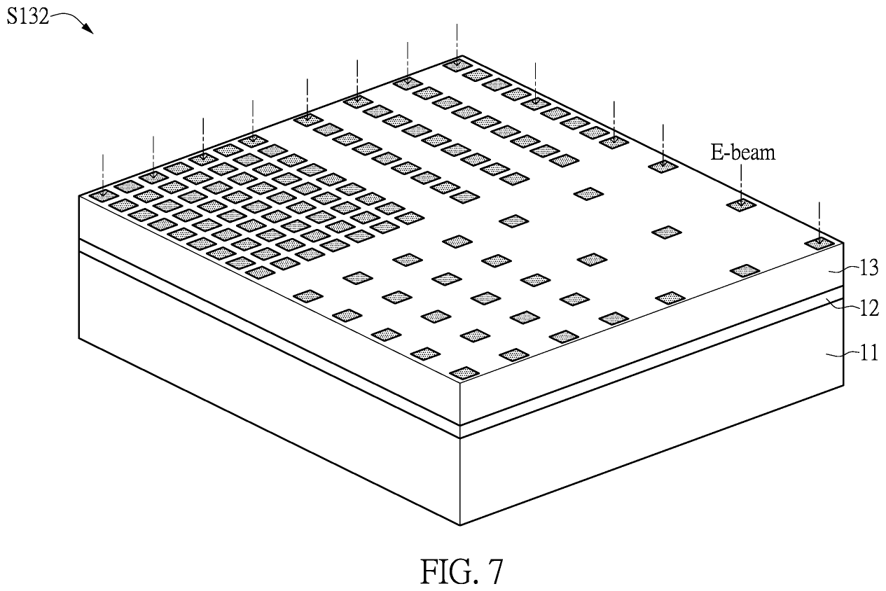

Referring to FIG. 1 to FIG. 5, a first embodiment of the present disclosure provides a method of manufacturing a plurality of optical filters with Fabry-Pérot (F-P) cavity structures. The method includes: Step S11: Providing a carrier 11 (as shown in FIG. 1). Step S12: Disposing a first reflection layer 12 on the carrier 11 (as shown in FIG. 2). Step S13: Disposing a first pattern optical layer 131 and a second pattern optical layer 132 on the surface of the first reflection layer 12. The first pattern optical layer 131 includes a plurality of first nanostructures 1311 and a second pattern optical layer 132 includes a plurality of second nanostructures 1321 (as shown in FIG. 3). Step S14: Reflowing the first pattern optical layer 131 and the second pattern optical layer 132 to form a first film layer 133 and a second film layer 134 (as shown in FIG. 4). Step S15: Coating a second reflection layer 15 on the first film layer 133 and the second film layer 134 (as shown in FIG. 5).

As shown in FIG. 3, a first pitch P1 being formed between two adjacent island-like first nanostructures 1311, a second pitch P2 being formed between two adjacent island-like second nanostructures 1321, The first pitch P1 and the second pitch P2 are the same but a first width/diameter of the first nanostructure 1311 and a second width/diameter of the second nanostructure 1321 are different from each other. Furthermore, the plurality of first nanostructure 1311 and the plurality of the second nanostructure 1321 has at least one difference in shape, size or quantity. In other embodiments, the first pitch P1 and the second pitch P2 are different from each other.

As shown in FIG. 4, the thicknesses of the first film layer 133 and the second film layer 134 can be precisely controlled by the reflow process of the patterned structure, such as the first pattern optical layer 131 and a second pattern optical layer 132. In the other hand, as transmission responds over visible and near infra-red wavelength, the thicknesses of the thin film structure are within a range of from 92 to 228 nm, preferably.

In addition, the carrier 11 is a silicon carrier 11 or a silicon-containing carrier 11. The first reflection layer 12 and the second reflection layer 15 are made of silver (Ag)., the first pattern optical layer 131 and the second pattern optical layer 132 are dielectric layers made of polymethyl methacrylate (PMMA). Thus, multispectral filters structures on the Ag-PMMA-Ag in a single chip (Quartz) are performed.

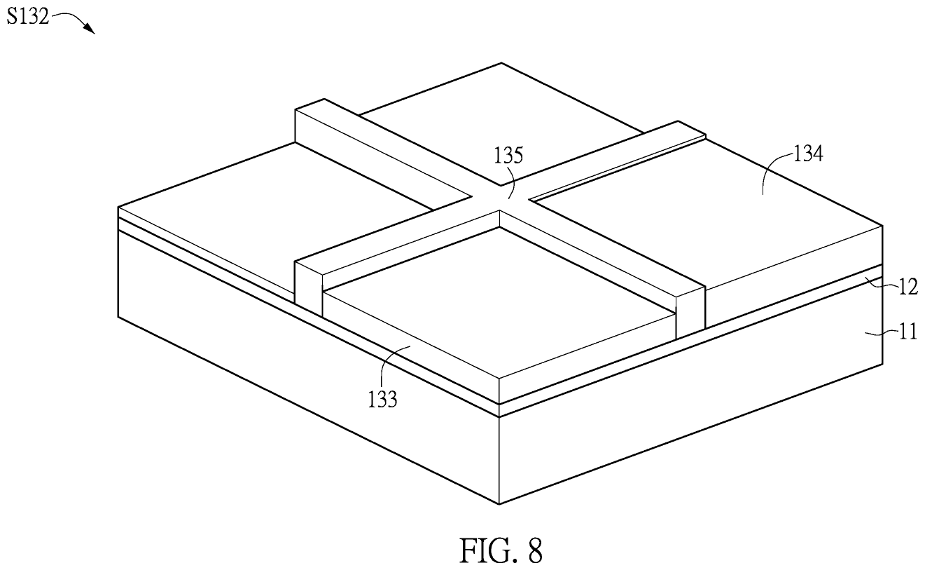

Referring to FIG. 6 to FIG. 8, simple-step-binary-lithography (SSBL) method is adopted. The step S13 for disposing the pattern optical layers can be further includes: S131: Coating an optical layer 13 on the first reflection layer 12. The optical layer 13 is an intermediate dielectric layer, such as PMMA. S132: Patterning the optical layer 13 to form the first pattern optical layer 131 and a second pattern optical layer 132 by electron beam lithography. Thus, a plurality of nano-patterned structures (hole-like) are developed within the plurality of patterning optical regions corresponding to the first pattern optical layer 131 and a second pattern optical layer 132. Then, a reflow process (step S14) to form the first film layer 133 and the second film layer 134.

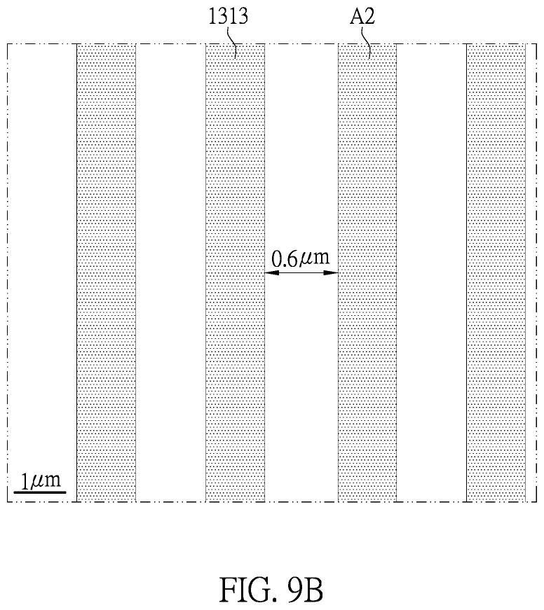

In addition, the step S132 further including: Forming an inter-boundary 135 between the first pattern optical layer 131 and a second pattern optical layer 132. The inter-boundary 135 is integrated by the first sidewall 1312 and the second sidewall 1322. The inter-boundary 135 is not reflowing during the reflowing process (as shown in FIG. 8). And the step S132 also can include: Forming a frame 136 around the first film layer 133 and a second film layer 134. The frame 136 is also integrated by the first sidewall 1312 and the second side wall 1322 (as shown in FIGS. 3 and 4).

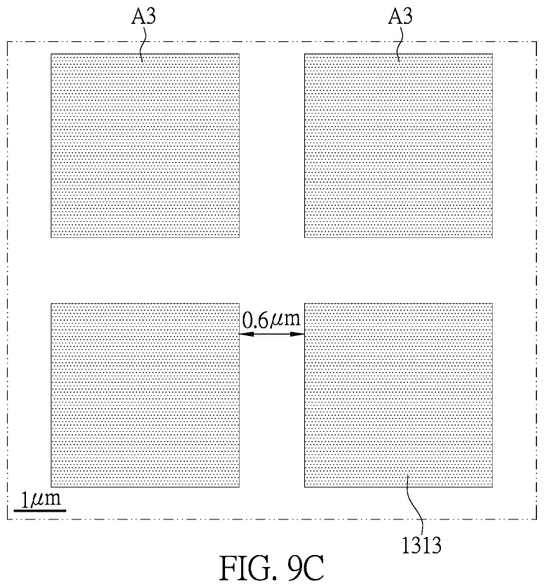

As shown as in FIGS. 9A-9C, according to the embodiment, a range of the pitch P1 or pitch P2 is from 0.5 to 1 μm. The nanostructures (the first nanostructures 1311 or the second nanostructures 1321) are hole-like or island-like. Referring to an area A1 of a square hole of 0.6 μm×0.6 μm is formed by the nanostructures 1313 an area A2 formed by the nanostructures 1313 is aisle-like And according to the other embodiment, the pitch p being formed between two adjacent areas A3 forming by the nanostructures is 0.6 μm.

The first optical film layer 131 and the second optical film layer 132 have different predetermined thicknesses, such that different color pixels are generated.

Please refer to FIG. 5 again, the present disclosure also provides a multispectral transmission filter array. The multispectral transmission filter array comprising: a carrier 11, a first reflection layer 12, an optical layer 13 and a second reflection layer 15. A first color pixels and a second color pixels forming by the optical layer 13 with difference thickness, and interpixel boundary 135 be formed between thereof.

According to the embodiment, a thickness of the interpixel boundary 135 is higher than the thickness of the first color pixels and the second color pixels. Furthermore, the optical layer 13 further comprises a frame 136 around the first color pixels and the second color pixels, and a thickness of the frame 136 is higher than that of the first color pixels and the second color pixels.

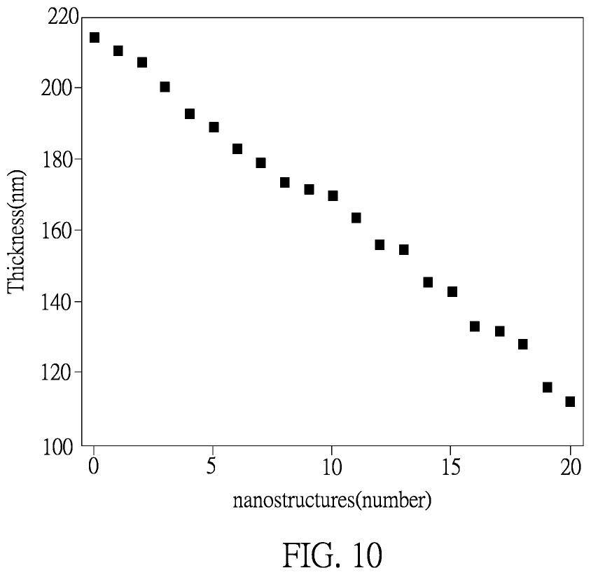

Please refer to FIG. 10, according to an embodiment of the present disclosure, the thickness of the optical layer 13 is inversely proportional to the number of nanostructures (corresponding to the first color pixels or the second color pixels). In other words, the greater the number of nanostructures, the thickness of the optical layer 13 will be thinner. Furthermore, in other embodiment, a thickness of the first color pixels 133 and the second color pixels 134 are inversely to wavelength. A thickness of a first film layer 133 corresponding to the first color pixels and a second film layer 134 corresponding to the second color pixels are within a range of from 92 to 228 nm, and a color filter range of the multispectral transmission filter array is between 450 to 850 nm.

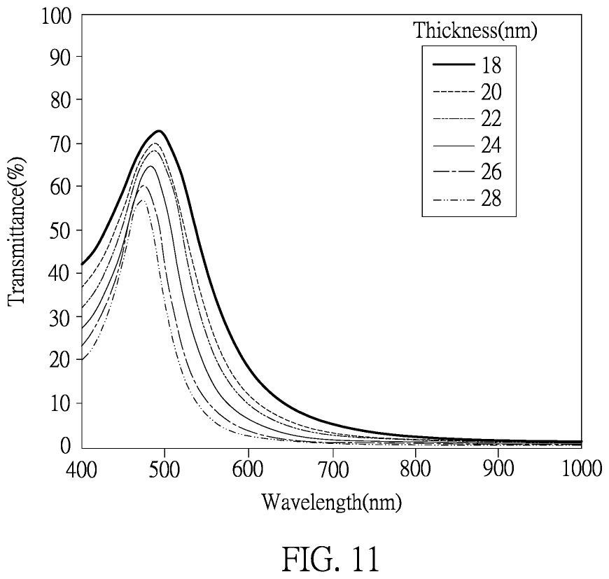

Please refer to FIG. 11, showing the relationship between thickness of different reflection layer, transmittance and wavelength. As the thickness of Ag layer was increased 18-28 nm, the transmission band became narrower with a drop in transmission peak from 80 to 65%. Here, a narrow band transmission with a 70% transmission peak is preferable if the 24-nm-thick Ag layer are used as metal reflector in the suggested F-P cavity structure. And in these embodiments, the first reflection layer 12 and the second reflection layer 15 have the same thickness. At the same wavelength, the smaller the thickness of the reflection layer would have the higher transmittance.

According to some embodiments, the optical layer 13 is selected form PMMA, SiO₂ and the carrier 11 is selected form SiO₂ or Si₃N₄.

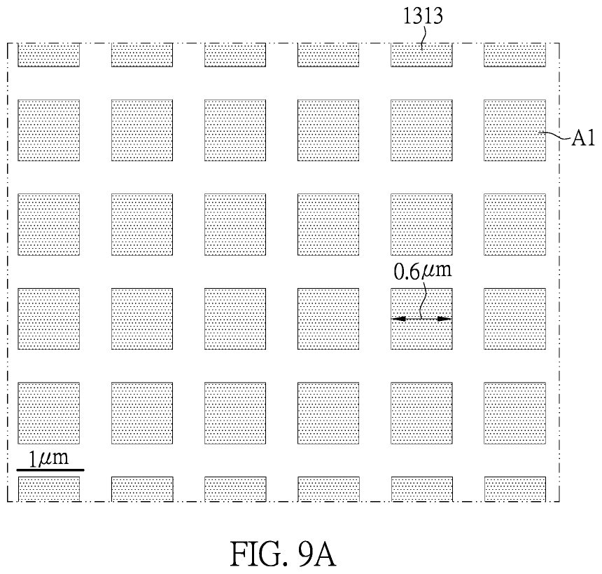

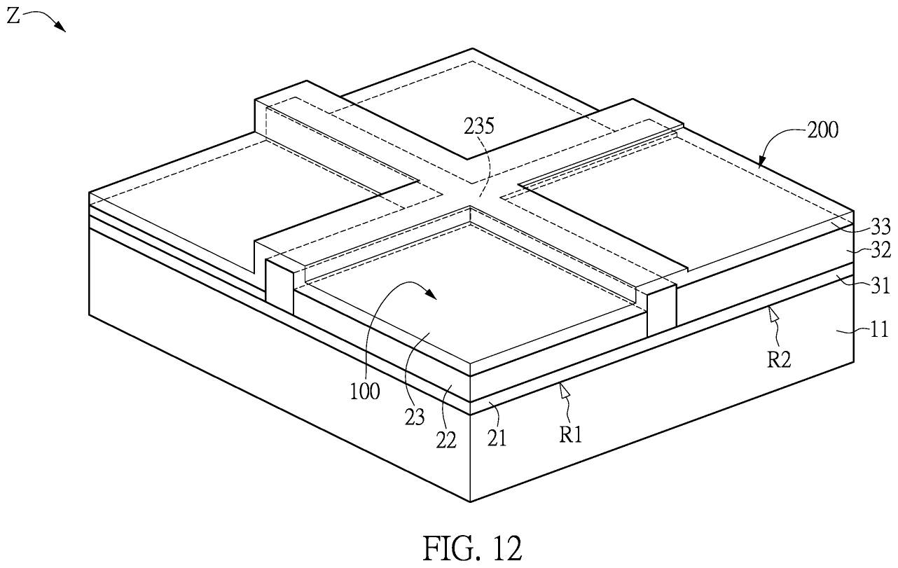

Refer to FIG. 12 and refer to FIGS. 7-8 again, the present disclosure also provides a multispectral transmission filter structure Z, comprising: a carrier 11, a first transmission filter 100, and a second transmission filter 200. The carrier 11 having a first sensing region R1 and a second sensing region R2. The first transmission filter 100 including a first bottom reflection layer 21, a first optical structure 22, and a first top reflection layer 23. The first bottom reflection layer 21 disposing on the first sensing region R1. The first optical structure 22 includes a first recess with a first depth. The first top reflection layer 23 disposing on the first optical structure 22. The second transmission filter 200 adjacent to the first filter sensor, the second transmission filter 200 including a second bottom reflection layer 31, a second optical structure 32 and a second top reflection layer 33. The second bottom reflection layer 31 disposing on the second sensing region R2. The second optical structure 32 includes a second recess with a second depth. The second top reflection layer 33 disposing on the second optical structure 32. The first depth and the second depth are different.

According to some embodiments, the first optical structure 22 and the second optical structure 32 are connected each other and formed as a wall 235 surrounding the first transmission filter 100 and the second transmission filter 200.

According to some embodiments, the first optical structure 22 receives a first wavelength the second optical structure 32 receives a second wavelength, the first wavelength is higher than the second wavelength, the second depth is deeper than the first depth.

According to some embodiments, the thicknesses of the plurality of pixels are within a range of from 92 to 228 nm, and a color filter range of the multispectral filter structure Z is between 450 to 850 nm.

According to some embodiments, the first optical structure 22 and the second optical structure 32 are selected form PMMA, SiO₂, the carrier 11 is selected form SiO₂, Si₃N₄, the first top reflection layer 23 and the second top reflection layer 33 are selected form DBR and Ag. And the first bottom reflection layer 21 and the second bottom reflection layer 31 are selected form DBR, and Ag. For example, the Ag-PMMA-Ag-SiO2 structure or the Ag-PMMA-DBR-Si₃N₄ can be considered as F-P cavity structure and the optical filters span both visible to infra-red range of wavelength.

### Beneficial Effects of the Embodiments

Therefore, in the multispectral transmission filter array provided by the present disclosure, by virtue of “an optical layer, disposing on the first reflection layer, wherein a first pixels, a second pixels, formed by the optical layer with difference thickness, and interpixel boundary formed between thereof”, the multispectral transmission filter array could be applied on a chip.

In addition, adjusting the thickness of the optical layer is equivalent to adjusting thickness of Fabry-Pérot (F-P) cavity structures which will modulate peak wavelength of optical filter. For example, the optical filter can span both visible to near infra-red.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method of manufacturing a plurality of optical filters with Fabry-Pérot (F-P) cavity structures, comprising:
providing a carrier;
disposing a first reflection layer on the carrier;
disposing a first pattern optical layer and a second pattern optical layer on the surface of the first reflection layer; wherein the first pattern optical layer includes a plurality of first nanostructures and a second pattern optical layer includes a plurality of second nanostructures,
reflowing the first pattern optical layer and the second pattern optical layer to form a first film layer and a second film layer; and
coating a second reflection layer on the first film layer and the second film layer.

2. The method according to claim 1, wherein a first pitch being formed between two adjacent first nanostructures, a second pitch being formed between two adjacent second nanostructures; and
wherein the first pitch and the second pitch are the same; wherein the first nanostructure and the second nanostructure are different from each other in shape or size.

3. The method according to claim 1, wherein the thicknesses of the first film layer and the second film layer are different and within a range of from 92 to 228 nm.

4. The method according to claim 1, the step for disposing the first and second pattern optical layers includes:
coating an optical layer on the first reflection layer; and
patterning the optical layer to form the first pattern optical layer and a second pattern optical layer by electron beam lithography.

5. The manufacturing method according to claim 4, further comprising forming an inter-boundary between the first pattern optical layer and a second pattern optical layer.

6. The manufacturing method according to claim 5, wherein the inter-boundary is not reflowing during the reflowing process.

7. The manufacturing method according to claim 1, wherein the nanostructures are hole-like or island-like.

8. The manufacturing method according to claim 1, wherein the first film layer and the second film layer have different predetermined thicknesses, such that different color pixels are generated.

9. A multispectral transmission filter array, comprising:
a carrier;
a first reflection layer, disposing on the carrier;
an optical layer, disposing on the first reflection layer, wherein the optical layer includes a plurality of color pixels with difference thickness and interpixel boundary formed between thereof; and
a second reflection layer, disposing on the plurality of color pixels.

10. The multispectral transmission filter array according to claim 9, wherein a thickness of the interpixel boundary is higher than the thickness of the plurality of color pixels.

11. The multispectral transmission filter array according to claim 9, wherein the optical layer further comprises a frame around the plurality of color pixels, and a thickness of the frame is higher than that of the plurality of color pixels.

12. The multispectral transmission filter array according to claim 9, wherein a thickness of the first reflection layer and second reflection are between 18-24 nm.

13. The multispectral transmission filter array according to claim 9, wherein a thickness of the plurality of color pixels are inversely to wavelength.

14. The multispectral transmission filter array according to claim 9, wherein the thicknesses of the plurality of color pixels are within a range of from 92 to 228 nm, and a color filter range of the multispectral transmission filter array is between 450 to 850 nm.

15. The multispectral transmission filter array according to claim 9, wherein the optical layer is selected form PMMA, SiO₂ and the carrier is selected form SiO₂, Si₃N₄.

16. A multispectral transmission filter structure, comprising:
a carrier having a first sensing region and a second sensing region;
a first transmission filter including:
a first bottom reflection layer disposing on the first sensing region;
a first optical structure includes a first recess with a first depth;
a first top reflection layer disposing on the first optical structure; and
a second transmission filter adjacent to the first filter sensor, including:
a second bottom reflection layer disposing on the second sensing region;
a second optical structure includes a second recess with a second depth;
a second top reflection layer disposing on the second optical structure;
wherein the first depth and the second depth are different.

17. The multispectral transmission filter structure according to claim 16, wherein the first optical structure and the second optical structure are connected each other and formed as a wall surrounding the first transmission filter and the second transmission filter.

18. The multispectral transmission filter structure according to claim 16, wherein the first optical structure receives a first wavelength the second optical structure receives a second wavelength, the first wavelength is higher than the second wavelength, the second depth is deeper than the first depth and a color filter range of the multispectral transmission filter structure is between 450 to 850 nm.

19. The multispectral transmission filter structure according to claim 18, wherein a thickness of the first top and second top reflection layers, and the second bottom and second bottom reflection layers are between 18-24 nm.

20. The multispectral transmission filter structure according to claim 16, wherein the first optical structure and the second optical structure are selected form PMMA, SiO₂, the carrier is selected form SiO₂, Si₃N₄, the first top and second top reflection layers are selected form Ag and DBR and the second bottom and second bottom reflection layers are selected form DBR and Ag.
